(19) United States
(12) Patent Application Publication
(10) Pub. No.: US 2025/0213480 A1
(43) Pub. Date: Jul. 3, 2025

(54) CONTINUOUS PROCESS

(71) Applicant: Micropore Technologies Limited, Redcar (GB)

(72) Inventors: Bruce Williams, Redcar (GB); David Hayward, Redcar (GB)

(73) Assignee: Micropore Technologies Limited, Redcar (GB)

(21) Appl. No.: 18/998,271

(22) PCT Filed: Jul. 25, 2023
(86) PCT No.: PCT/GB2023/051955

§ 371 Date: Jan. 24, 2025

(30) Foreign Application Priority Data
Jul. 25, 2022 (GB) 2210829.4

(51) Int. Cl. A61K 9/1277

(52) U.S. Cl. CPC A61K 9/1277

(57) ABSTRACT

There is described a continuous process for the preparation of microscopic particles. e.g. nanoparticles or microparticles, said process comprising the steps of: (i) controlling provision of a first liquid phase to a first membrane, the first membrane defining a first plurality of apertures: (ii) controlling provision of a second liquid phase to the first membrane via the first plurality of apertures to form a mixture; and controlling provision of the mixture to a second membrane to form a stabilised suspension of particles; concentrating the suspension of particles and controlling provision of a pH buffer to a third membrane; and controlling provision of the stabilised mixture to a fourth membrane for microfiltration, to isolate the microscopic particles.

### FIELD OF THE INVENTION

The present invention relates to a continuous process for the production of microparticles or nanoparticles.

More particularly, the invention relates to a continuous process for the production of microparticles or nanoparticles, especially Lipid Nanoparticles (LNPs).

### BACKGROUND TO THE INVENTION

Microparticles and nanoparticles have important applications in biomedicine, pharmacy, medicine, cosmetics, chemical industries, agriculture, veterinary science, etc.

Many techniques are available for the manufacture of microparticles and nanoparticles. Such techniques include precipitation or co-precipitation of sparingly soluble products from aqueous or non-aqueous solutions, Sol-Gel processing and the use of microemulsions.

International patent application No. WO 2022/018441 describes a method of preparing lipid vesicles, Liposomes or lipid nanoparticles (LNPs) utilising membrane emulsification techniques.

Membrane emulsification techniques offer advantages in the preparation of lipid vesicles and microparticles and nanoparticles in general.

However, most commercially available techniques for the preparation of microparticles and nanoparticles utilise a batch process. A batch process is a process whereby during the operation, no materials enter or leave the system during the operational period. Generally, material is left in the system, material accumulation, and the reaction vessel must be cleaned between each batch.

Examples of the use of batch processes include use in; beverage processing, dairy products, pharmaceutical formulations and soap manufacturing.

The use of batch processes tend to limit the scale of manufacturing, and are labour intensive. Further plant will generally be idle due to changes of batches and cleaning between batches. It may also be difficult to maintain quality in a product as there may be variations from one batch to another.

In the case of fed batch processes, often used in biotechnology, there can also be significant differences in residence time of particles in the process, leading to differences in quality. For example tangential flow filtration is often operated in a fed batch process. Product is gradually introduced to a circulating loop and concentrated, followed by further recirculation whilst a diafiltration buffer is gradually introduced. Clearly those particles introduced at the start have a longer residence time than those introduced at the end.

Therefore, there is a need for a continuous process technique for the manufacture of microparticles and nanoparticles.

### SUMMARY OF THE INVENTION

We have now found a novel, continuous process for the manufacture of microscopic particles, e.g. microparticles and nanoparticles, by using membrane emulsification apparatus and techniques.

Thus, according to a first aspect of the invention there is provided a continuous process for the preparation of microscopic particles, e.g. nanoparticles or microparticles, said process comprising the steps of:
(i) controlling provision of a first liquid phase to a first membrane, the first membrane defining a first plurality of apertures;
(ii) controlling provision of a second liquid phase to the first membrane via the first plurality of apertures to form a mixture;
(iii) controlling provision of the mixture to a second membrane, the second membrane defining a second plurality of apertures;
(iv) controlling provision of a pH buffer liquid phase to the second membrane via the second plurality of apertures to form a stabilised suspension of particles;
optionally:
(v) concentrating the suspension of particles and controlling provision of the pH buffered mixture to a third membrane, the third membrane defining a third plurality of apertures;
(vi) controlling provision of a concentrated suspension phase to the third membrane via the third plurality of apertures to remove solvent, and exchange the particles into an excipient buffer; and
(vii) controlling provision of the stabilised mixture to a fourth membrane for microfiltration, to isolate the microscopic particles.

It will be understood by the person skilled in the art that one or more of steps (v) and (vi) may be optional.

It will also be understood by the person skilled in the art that it may be possible to use a non-buffered solution, since much of the stabilisation is thought to come from ethanol dilution.

In step (v) the concentration of the suspension of particles can be achieved using single pass tangential flow filtration cassette equipped with an ultrafiltration or microfiltration membrane suitably sized to retain the particles. For larger microparticles an AXF® cross-flow apparatus (available from Micropore Technologies Limited) equipped with membranes comprising slotted apertures may be used. Such slotted aperture membranes are described in US patent application No. US 2009/211991, which is incorporated herein by reference.

Generally, in the continuous process of the invention the sequence of steps may be considered to comprise:
Formation of microscopic particles
Dilution/adjustment of the microscopic particles
Concentration of the microscopic particles
Dilution of the microscopic particles with a diafiltration buffer
Concentration
Dilution with diafiltration buffer

Further repeats of these steps may be carried out to achieve a desired composition, stopping either after concentration or dilution.

When the continuous process of the invention relates to lipid vesicles, e.g. lipids or LNPs, the mixture may comprise a lipid vesicle suspension. When the continuous process of the invention relates to conventional microparticles, the mixture may comprise an emulsion, e.g. a solid in liquid emulsion or liquid/liquid emulsion.

For nanoparticles the continuous process of the invention may include an optional final step which includes an inline sterile filtration to remove bioburden.

In the continuous process of the invention any number of concentration-dilution steps may be included. Typically, there will be at least 2 concentration-dilution steps in order to achieve sufficiently low solvent concentrations, but this could be 3 or more.

For a continuous process, steps (i) to (vii) may preferably be carried out sequentially with or without an intervening isolation or purification step. Furthermore, intermediate holding vessels, residence coils, pumps, and the like, may be utilised.

For example if solvent diffusion is slow (e.g. through large particles of low porosity) then extra residence time may be required. Splitting the sequence may also allow cheaper low pressure pumps to be used.

For the avoidance of doubt the term “microscopic particles” shall include nanoparticles, microparticles, microspheres, microcapsules, etc.

Each of the membranes, e.g., the first, second, third and fourth membranes, may be the same or different. Generally, each membrane defines a plurality of pores and may be substantially tubular or cylindrical in shape and may have a first end defining a first inlet aperture and a second outlet aperture. . . . Thus, each of the membranes, which may be the same or different, may comprise a laboratory dispersion cell (LDC), which uses a precision engineered circular membrane, with a stirrer being used to generate the shear required for droplet formation; or a crossflow apparatus (AXF®).

It will be understood by the person skilled in the art that the number of membranes used in the continuous process may vary, depending, inter alia, upon the nature of the material being processed, etc. Generally, the number of membranes used in the continuous process of the invention is from about 1 to about 4. To minimise solution volumes for the buffer exchange/washing more stages may be used, each with smaller volume of fresh buffer. Capital cost increases but solution costs decrease. For example, for microparticles if a 3 stage diafiltration is used, each stage with one dilution membrane and one filtration membrane, that would give 7 stages including the droplet generation membrane.

For each of the tubular or cylindrical membranes, the internal diameter of the membrane may be varied. Generally, the internal diameter of the membrane will be fairly small.

Alternatively, one or more of the membranes, e.g. one or more of the first, second, third and fourth membranes, may comprise a crossflow membrane apparatus (AXF®). According to one aspect of the invention at least one of the membranes comprises a crossflow apparatus (AXFR). According to another aspect of the invention all of the membranes comprise crossflow apparatus (AXFR). A crossflow membrane apparatus uses the flow of, e.g. a continuous phase, to sweep and evenly mix flows of, e.g. a disperse phase coming through the membrane pores.

It will be understood that some or all of the membranes used may comprise crossflow membranes. The continuous process of the present invention may use a mixture conventional tubular membranes, such as laboratory dispersion cells (LDCs) and crossflow membranes. In a preferred embodiment, all of the membranes used in the continuous process of the invention comprise crossflow membranes.

In one aspect of the present invention the continuous process may comprise the preparation of microscopic lipid vesicles, e.g. liposomes or lipid particles, more specifically, liposomes or lipid nanoparticles (LNPs).

Lipid vesicles, e.g. LNPs, are especially useful as drug delivery carriers and in encapsulating a broad variety of nucleic acids (RNA and DNA); and as such, they are the most popular non-viral gene delivery system, for example, used in vaccine delivery.

Processes for the preparation of lipid vesicles using membrane emulsification techniques are described in International patent application No. WO 2022/018441, which is incorporated herein by reference.

According to this aspect of the invention step (i) of the continuous process may comprise controlling provision of a first liquid phase to a first membrane, wherein the first liquid phase comprises a lipid phase; and the second liquid phase comprises an aqueous phase. In particular, the aqueous phase may comprise one or more therapeutically active agents, such as, DNA and RNA, e.g. mRNA. According to one aspect of the invention the lipid vesicles are liposomes. According to another aspect of the invention the lipid vesicles are LNPs.

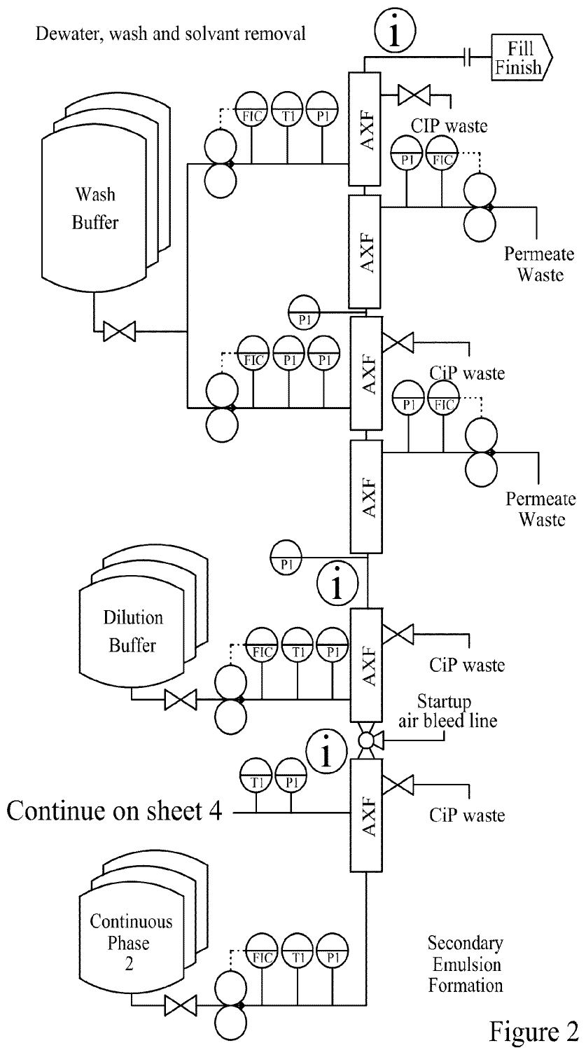

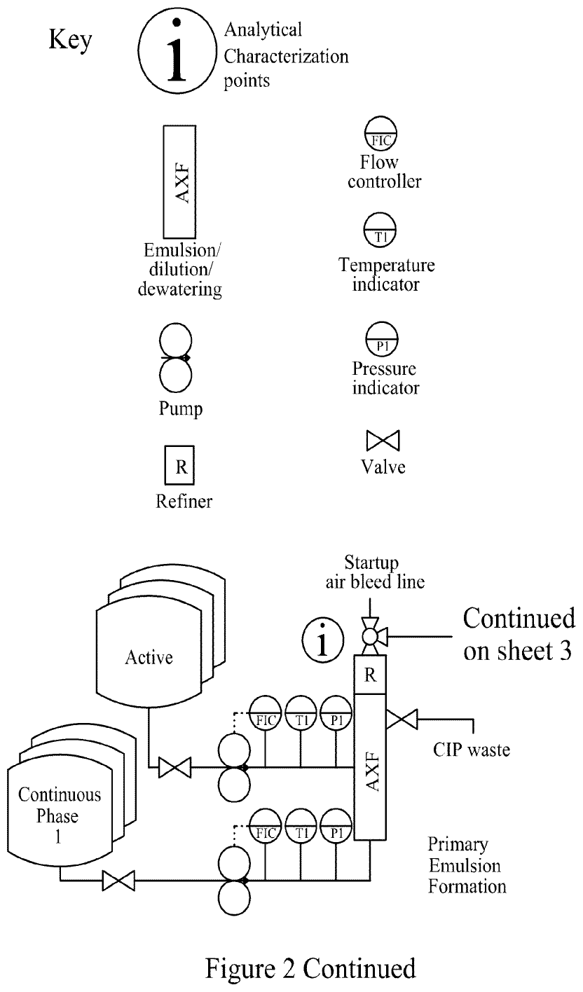

The active agent may be dispersed in water droplets within a polymer solution in solvent droplets. Such a dispersion of an active agent may thus comprise a “primary emulsion” (as illustrated in FIG. 2 herein).

In this aspect of the invention the product of the continuous process of preparing lipid vesicles is a lipid vesicle composition comprising of a lipid bilayer encapsulating an aqueous core. The aqueous core may include one or more active agents or the lipid vesicles may be produced unloaded and loaded afterwards (active loading). Loading of active agents can be attained either by passive loading i.e. the active agent is encapsulated during formation of the lipid vesicle; or active loading, i.e. the active agent is loaded after formation of the lipid vesicle.

Thus, according to one aspect of the invention the lipid vesicles are produced loaded (passive loading).

According to another aspect of the invention the lipid vesicles are produced unloaded and loaded afterwards (active loading).

When the lipid vesicles are liposomes then the solvent phase may comprise an aqueous phase. When the lipid vesicles comprise LNPs the solvent phase may comprise a non-aqueous solvent phase.

Lipid vesicles and liposomal particles are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter 100≤nm; and LUVs have a diameter >100 nm.

Lipid vesicles of the present invention may preferably be SUVs or LUVs with a diameter in the range of 50-220 nm. For a composition comprising a population of SUVs or LUVs with different diameters: (i) at least 80% by number should have diameters in the range of 20-220 nm; (ii) the average diameter of the population is ideally in the range of 40-200 nm, and/or (iii) the diameters should have a polydispersity index (PDI)≤0.3, e.g. from about 0.02 to about 0.3, preferably between 0.02 and 0.2. The lipid vesicle may be substantially spherical.

According to a further aspect of the invention the microscopic particles are microparticles or nanoparticles as herein described, having applications in biomedicine, pharmacy, medicine, cosmetics, chemical industry, agriculture, veterinary science, etc., Such microscopic particles will usually include a chemically or biologically active substance. Thus, the continuous process of the invention may comprise preparing microscopic particles including a chemically or biologically active substance, said method comprising controlling provision of a liquid phase, wherein said liquid phase comprises a solution of the compound, in a first flow direction to a membrane, said membrane defining a plurality of pores; and controlling the liquid phase after it has passed through the membrane via the plurality of pores, to form microparticles comprising a chemically or biologically active substance.

According to a yet further aspect of the invention the microscopic particles are solidified particles, e.g. crystalline particles. Thus, the continuous process of the invention may comprise preparing solid particles of a compound, said method comprising controlling provision of a liquid phase, wherein said liquid phase comprises a solution of the compound, in a first flow direction to a membrane, said membrane defining a plurality of pores; and controlling the supersaturation of the liquid phase after it has passed through the membrane via the plurality of pores, to form solid particles of the compound. Such solid particles are described in our co-pending International patent application WO 2022/023759 which is incorporated herein by reference.

Step (iv) of the continuous process of the invention may comprise formation of a pH buffered mixture by controlling provision of the mixture to a second membrane and controlling provision of a pH adjustment buffer to form a stabilised mixture.

The choice of loading of lipid vesicles, i.e. active or passive loading of liposomes or LNPs, may influence the choice of aqueous phase pH adjustment buffer.

Batch tangential flow filtration tends to last hours with a circulating pump and moderate shear. Reducing residence time to minutes and making single pass should be much more gentle and reduce losses.

The continuous process apparatus of the invention may include one or more analytical characterisation points. Thus, for example, an analytical characterisation point may be included at or towards the end of each step of the continuous process or each pair of steps of the continuous process, e.g. at or towards the end one or more of steps (ii), (iv) and (vi). It will be understood by the person skilled in the art that such analytical characterisation points may be situated at or adjacent to other suitable points in the continuous process of the invention.

Techniques for preparing suitable lipid vesicles are well known in the art. One such method involves mixing an ethanolic solution of the lipids with an aqueous solution of the active agent. It will be understood by the person skilled in the art that since the technique relies on solvent-water miscibility, other water miscible solvents may suitably be used, for example, C₁-C₆ alkanols, such as, methanol, ethanol, propanol, butanol, pentanol, hexanol, and the like.

The method of the present invention is adaptable to large-scale, commercial production of formulations of nanoscale lipid vesicles, particularly of those that comprise substantially homogenous lipid vesicle particle sizes that may be no bigger than about 220 nm in diameter. For example, more than 90% (volume weighted, e.g. as determined by dynamic light scattering) of lipid vesicles are less than about 220 nm; or more than 99% less than about 220 nm. Such sized particles can be readily filter sterilised according to industry-approved clinical manufacturing standards and or GMP (Good Manufacturing Practice).

When at least one or more of the membranes used in the continuous process of the invention comprises a crossflow membrane apparatus (AXF®); said crossflow emulsification apparatus may comprise:
an outer tubular sleeve provided with a first inlet at a first end; a lipid vesicle outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with a chamfered region; the chamfered region is provided with a plurality of orifices and a furcation plate; and
controlling provision of the first liquid phase to the tubular membrane; and controlling provision of a second liquid phase to the tubular membrane via the plurality of pores to form a lipid vesicle suspension.

In the method of the present invention the crossflow membrane emulsification uses the flow of a continuous phase, to sweep and evenly mix flows of a disperse phase coming through the membrane pores. The mixing or micromixing comprises a controlled mixing of phases.

In one aspect of the invention the crossflow apparatus includes an insert as herein described and the first inlet is a continuous phase first inlet and the second inlet is a disperse phase inlet; such that the disperse phase travels from outside the tubular membrane to inside.

In another aspect of the invention the crossflow apparatus does not include an insert and the first inlet is a disperse phase first inlet and the second inlet is a continuous phase inlet; such that the disperse phase travels from inside the tubular membrane to outside.

In another aspect of the invention the disperse phase is the solvent phase and the continuous phase is a lipid phase. The solvent phase may optionally include one or more active agents as herein defined.

In one aspect of the invention the disperse phase is the lipid phase and the continuous phase is a solvent phase. The solvent phase may optionally include one or more active agents as herein defined.

When an insert is present and the tubular membrane is positioned inside the outer sleeve, the spacing between the insert and the tubular membrane may be varied, depending upon the laminar conditions desired, etc. Generally, the insert will be located centrally within the tubular membrane, such that the spacing between the insert and the membrane will comprise an annulus, of equal or substantially equal dimensions at any point around the insert. Thus, for example, the spacing may be from about 0.05 to about 10 mm (distance between the outer wall of the insert and the inner wall of the membrane), from about 0.1 to about 10 mm, from about 0.25 to about 10 mm, or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

When the tubular membrane is positioned inside the outer sleeve, the spacing between the tubular membrane and the outer sleeve may be varied. Generally, the tubular membrane will be located centrally within the outer sleeve, such that the spacing between the membrane and the sleeve will comprise an annulus, of equal or substantially equal dimensions at any point around the tubular membrane. Thus, for example, the spacing may be from about 0.5 to about 10 mm (distance between the outer wall of the membrane and the inner wall of the sleeve), or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

In an alternative embodiment of the invention the insert is tapered, such that the spacing between the insert and the tubular membrane may be divergent along the length of the membrane. The spacing and the amount of divergence varied, depending upon the gradient of the tapered insert, the laminar conditions/flow velocities desired, size distribution, etc. It will be understood by the person skilled in the art that depending upon the direction of taper, the spacing between the insert and the tubular membrane may be divergent or convergent along the length of the membrane. The use of a tapered insert may be advantageous in that a suitable taper may allow the laminar flow to be held constant for a particular formulation and set of flow conditions. Thus, the tapered insert may be used to control variation in mixing conditions resulting from changes in fluid properties, such as viscosity, as the ethanol or other solvent and lipid concentration increases through its path along the length of the membrane.

In an alternative embodiment of the invention, when used, the crossflow apparatus may comprise more than one tubular membrane located inside the outer tubular sleeve, i.e. a plurality of tubular membranes. When a plurality of tubular membranes is provided, each membrane may optionally have an insert, as herein described, located inside it. A plurality of membranes may be grouped as a cluster of membranes positioned alongside each other. Desirably the membranes are not in direct contact with each other. It will be understood that the number of membranes may vary depending upon, inter alia, the nature of the materials to be produced.

Thus, by way of example only, when a plurality of tubular membranes is present, the number of membranes may be from 2 to 100.

The inclined second inlet provided in the outer tubular sleeve will generally comprise a branch of the tubular sleeve and may be perpendicular to the longitudinal axis of the tubular sleeve. The position of the branch or second inlet may be varied and may depend upon the plane of the membrane. In one embodiment the position of the branch or second inlet will be substantially equidistant from the inlet and the outlet, although it will be understood by the person skilled in the art that the location of this second inlet may be varied. It is also within the scope of the present invention for more than one branch inlet to be provided. For example the use of a dual branch may suitably allow for bleeding the continuous phase during priming, or flushing for cleaning, or drainage/venting for sterilisation.

The inlet and outlet ends of the outer sleeve will generally be provided with a seal assembly. Although the seal assemblies at the inlet and outlet ends of the outer sleeve may be the same or different, preferably each of the seal assemblies is the same. Normal O-ring seals involve the O-ring being compressed between the two faces on which the seal is required-in a variety of geometries. Commercially available O-ring seals are provided with different groove options with standard dimensions. Each seal assembly will comprise a tubular ferrule provided with a flange at each end. A first flange, located at the end adjacent to the outer sleeve (when coupled) may be provided with a circumferential internal recess which acts as a seat for an O-ring seal.

However, it will be understood by the person skilled in the art that other means of making seal may suitably be used, for example, use of a screwed fitting tightened to a particular torque which would avoid the need for close tolerances; or clamping parts to a particular force followed by welding (which may be particularly suitable when using a plastic crossflow apparatus).

The internal diameter of each the tubular membranes may be varied. In particular, the internal diameter of a tubular membrane may vary depending upon whether or not an insert is present and at what stage of the continuous process the membrane is applied. Generally, the internal diameter of the tubular membrane will be fairly small. In the absence of an insert the internal diameter of the tubular membrane may be from about 1 mm to about 10 mm, or from about 2 mm to about 8 mm, or from about 4 mm to about 6 mm. When the tubular membrane is intended for use with an insert, the internal diameter of the tubular membrane may be from about 5 mm to about 50 mm, or from about 10 mm to about 50 mm, or from about 20 mm to about 40 mm, or from about 25 mm to about 35 mm. Higher internal diameter of the tubular membrane may only be capable of being subjected to lower injection pressure. The upper limit of the internal diameter of the tubular membrane may depend upon, inter alia, the thickness of the membrane tube, since the cylinder needs to be able to cope with the external injection pressure, and whether it's possible to drill consistent holes through that thickness. The chamber inside the cylindrical membrane usually contains the continuous phase liquid.

In the membrane the pores may be uniformly spaced or may have a variable pitch. Alternatively, the membrane pores may have a uniform pitch within a row or circumference, but a different pitch in another direction.

The pores in the membrane may vary. By way of example only, the pores in the membrane may have a pore diameter of from about 1 μm to about 200 μm, or from about 1 μm to about 100 μm, or about 10 μm to about 100 μm, or about 20 μm to about 100 μm, or about 30 μm to about 100 μm, or about 40 μm to about 100 μm, or about 50 μm to about 100 μm, or about 60 μm to about 100 μm, or about 70 μm to about 100 μm, or about 80 μm to about 100 μm, or about 90 μm to about 100 μm. In a further embodiment of the invention the pores in the membrane may have a pore diameter of from about 1 μm to about 40 μm, e.g. about 3 μm, or from about 5 μm to about 20 μm, or from about 5 μm to about 15 μm.

In the membrane the shape of the pores may be substantially tubular or linear, e.g. slotted pores. For filtration for microparticle continuous process, slotted pores may be preferred. The aspect ratio of the slot may be such that it retains the product particle, but the particles are never able to be “sucked onto” and consequently block a pore, i.e. they will instead continue to flow past. When using a membrane with slotted pores the feed flows between the insert, e.g. the tapered insert, and the membrane, with permeate flowing through pores and out via the side port (second inlet).

However, it is within the scope of the present invention to provide a membrane with uniformly tapered pores. Such uniformly tapered pores may be advantageous in that their use may reduce the pressure drop across the membrane and potentially increase throughput/flux. It is also within the scope of the present invention to provide a membrane in which the diameter is essentially constant, but the internal bore is non-circular (for example rectangular slots) or convoluted (for example tapered or stepped diameter to minimise pressure drop), providing pores with a high aspect ratio.

The interpore distance or pitch may vary depending upon, inter alia, the pore size; and may be from about 1 μm to about 5,000 μm, or from about 1 μm to about 1,000 μm, or from about 2 μm to about 800 μm, or from about 5 μm to about 600 μm, or from about 10 μm to about 500 μm, or from about 20 μm to about 400 μm, or from about 30 μm to about 300 μm, or from about 40 μm to about 200 μm, or from about 50 μm to about 100 μm, e.g. about 75 μm.

The surface porosity of the membrane may depend upon the pore size and may be from about 0.001% to about 20% of the surface area of the membrane; or from about 0.01% to about 20%, or from about 0.1% to about 20%, or from about 1% to about 20%, or from about 2% to about 20%, or from about 3% to about 20%, or from about 4% to about 20%, or from about 5% to about 20, or from about 5% to about 10%.

The arrangement of the pores may vary depending upon, inter alia, pore size, throughput, etc. Generally, the pores may be in a patterned arrangement, which may be a square, triangular, linear, circular, rectangular or other arrangement. In one embodiment the pores are in a square arrangement.

It will be understood that the apparatus of the invention; and in particular the membrane, may comprise known materials, such as glass; ceramic; metal, e.g. stainless steel or nickel; polymer/plastic, such as a fluoropolymer; or silicon. The use of metals, such as stainless steel or nickel, or polymer/plastic, such as a fluoropolymer is advantageous in that, inter alia, the apparatus and/or membranes may be subjected to sterilisation, using conventional sterilisation techniques known in the art, including gamma irradiation where appropriate. The use of polymer/plastic material, such as a fluoropolymer, is advantageous in that, inter alia, the apparatus and/or membrane may be manufactured using injection moulding techniques known in the art.

As described herein an insert may be included in any one of the membranes to facilitate even flow distribution. However, it is within the scope of the crossflow apparatus of the present invention for the insert to be absent. When an insert is present, the furcation plate may be adapted to split the flow of continuous phase or the disperse phase into a number of branches. Whether the furcation plate splits the continuous phase or the disperse phase will depend upon the direction of flow of the continuous phase, i.e. whether the continuous phase flows through the first inlet or the second inlet. Although the number of furcation plates may be varied, the number selected should be suitable lead to even flow distribution and (at the lipid vesicle outlet end) not have excessive shear. Preferably, when the insert is present the furcation plate is a bi-furcation plate or a tri-furcation plate to provide a uniform continuous phase flow within the annular region between the insert and the membrane. Most preferably the furcation plate is a tri-furcation plate.

The number of orifices provided in the insert may vary depending upon the injection rate, etc. Generally the number of orifices may be from 2 to 6. Preferably the number of orifice is three.

The chamfered region on an insert is advantageous in that it enables the insert to be centred when it is located in position inside the membrane. The external circumference of the ends of the insert has a minimal tolerance with the internal diameter of the tubular membrane. This enables the insert to be accurately centred, thereby providing a consistent annulus leading to a consistent laminar flow. Generally, the chamfered region will comprise a shallow chamfer, which is advantageous in that it evens the flow distribution and allows the use of orifices in the insert with larger cross-sectional area than could be achieved if the flow simply entered through orifices parallel to the axis of the insert. This keeps the fluid velocity down and therefore minimises unwanted pressure losses, and shear on the outlet. The distance between the start of the orifices and the start of the porous region on the tubular membrane allows an even velocity distribution to be established. The radial dimension of the insert is selected to provide an annular depth to provide a certain laminar flow for the flowrates chosen. The axial dimension is designed to generally give a combined orifice area which is greater than both the annular area and the inlet/exit tube area.

The use of membrane emulsification techniques in the continuous preparation of lipid vesicles as herein described may comprise the use of turbulent flow, e.g. by stirring; or the use of laminar flow. In a particular aspect of the invention the continuous membrane emulsification technique comprises the use of laminar flow, i.e. whilst generally avoiding or minimising any turbulent flow.

The use of membrane emulsification techniques in the preparation of lipid vesicles as herein described may include the use of one or more pump systems. It will be understood that any conventionally known pumping system for use with membrane emulsification may suitably be used. However, in a particular aspect of the invention the pump system may comprise a gear pump or a peristatic pump; and combinations thereof.

The lipid vesicles thus obtained have a high reproducibility both in the encapsulation rate and in the particle size distribution (polydispersity). The lipid vesicles may have a polydispersity index of ≤0.3, e.g. from about 0.05 to about 0.3.

The continuous method of the invention can be used to precisely control the distribution of chemical conditions and mechanical forces so that they are constant on a length scale equivalent to that of a lipid vesicle. Hence, resultant lipid vesicle populations that are more uniform in size, hence of low polydispersity.

In one aspect of the invention when at least one crossflow apparatus is used, the crossflow apparatus may include an insert as herein described and the first inlet is a continuous phase first inlet and the second inlet is a disperse phase inlet; such that the disperse phase travels from outside the tubular membrane to inside.

In another aspect of the invention when at least one crossflow apparatus is used, the crossflow apparatus does not include an insert and the first inlet is a disperse phase first inlet and the second inlet is a continuous phase inlet; such that the disperse phase travels from inside the tubular membrane to outside.

Separation, purification and/or dilution of the lipid vesicles might also be performed by any suitable method. Preferably, in the continuous process of the invention lipid vesicles are filtrated, more preferably the lipid vesicles are separated or purified by filtration through a sterile filter. For active loading and/or RNA loading dilution, in order to reduce the solvent concentration or to replace buffers may be followed by concentration by ultrafiltration.

According to a further aspect of the invention there is provided apparatus for a continuous process for the preparation of microscopic particles as herein described, said apparatus comprising an array of membranes.

According to this aspect of the invention the array of membranes may comprise:
(i) a first membrane for formation of an mixture by controlling provision of a first liquid phase to the first and controlling provision of a second liquid phase to the first membrane;
(ii) a second membrane for formation of a pH buffered mixture by controlling provision of the mixture to a second membrane, the second membrane defining a second plurality of apertures and controlling provision of a pH buffer liquid phase to the second membrane via the second plurality of apertures;
(iii) a third membrane for formation of a stabilised mixture by controlling provision of the pH buffered mixture to the third membrane, the third membrane defining a third plurality of apertures and controlling provision of a diafiltration buffering liquid phase to the third membrane via the third plurality of apertures; and
(vii) a fourth membrane for isolation of the microscopic particles by controlling provision of the stabilised mixture to the fourth membrane for microfiltration.

Lipid vesicles, e.g. liposomes and lipid nanoparticles (LNPs), prepared by the method of the invention are useful as components in pharmaceutical compositions for immunising subjects against various diseases. These compositions will typically include a pharmaceutically acceptable carrier in addition to the lipid vesicle.

Therefore, according to a further aspect of the present invention there are provided lipid vesicles prepared by the continuous process herein described. According to one aspect of the invention the lipid vesicles prepared by the continuous process herein described are liposomes. According to another aspect of the invention the lipid vesicles prepared by the method herein described are LNPs.

According to this aspect of the invention the lipid vesicle may further include an active agent.

By way of example only, active agents for use in the lipid vesicles of the present invention include, but shall not be limited to, biologically active agents, such as pharmaceutically active agents, vaccines and pesticides. Biologically active compounds may also include, for example, a plant nutritive substance or a plant growth regulant. Alternatively, the active agent may be non-biologically active, such as, a plant nutritive substance, a food flavouring, a fragrance, and the like.

Pharmaceutically active agents refer to naturally occurring, synthetic, or semi-synthetic materials (e.g., compounds, fermentates, extracts, cellular structures) capable of eliciting, directly or indirectly, one or more physical, chemical, and/or biological effects, in vitro and/or in vivo. Such active agents may be capable of preventing, alleviating, treating, and/or curing abnormal and/or pathological conditions of a living body, such as by destroying a parasitic organism, or by limiting the effect of a disease or abnormality by materially altering the physiology of the host or parasite. Such active agents may be capable of maintaining, increasing, decreasing, limiting, or destroying a physiologic body function. Active agents may be capable of diagnosing a physiological condition or state by an in vitro and/or in vivo test. The active agent may be capable of controlling or protecting an environment or living body by attracting, disabling, inhibiting, killing, modifying, repelling and/or retarding an animal or microorganism. Active agents may be capable of otherwise treating (such as deodorising, protecting, adorning, grooming) a body. Depending upon the effect and/or its application, the active agent may further be referred to as a bioactive agent, a pharmaceutical agent (such as a prophylactic agent, or a therapeutic agent), a diagnostic agent, a nutritional supplement, and/or a cosmetic agent, and includes, without limitation, prodrugs, affinity molecules, synthetic organic molecules, polymers, molecules with a molecular weight of 2 kD or less (such as 1.5 kD or less, or 1 kD or less), macromolecules (such as those having a molecular weight of 2 kD or greater, preferably 5 kD or greater), proteinaceous compounds, peptides, vitamins, steroids, steroid analogues, nucleic acids, carbohydrates, precursors thereof and derivatives thereof. Active agents may be ionic, non-ionic, neutral, positively charged, negatively charged, or zwitterionic, and may be used singly or in combination of two or more thereof. Active agents may be water insoluble or water soluble.

The term “macromolecule” used herein refers to a material capable of providing a three-dimensional (e.g., tertiary and/or quaternary) structure.

A wide variety of pharmaceutically active agents may be utilised in the continuous process of the present invention. Thus, the pharmaceutically active agent may comprise one or more of a polynucleotide, a peptide, a protein, a small organic active agent, a small inorganic active agent and mixtures thereof.

A polynucleotide active agent may comprise one or more of an oligonucleotide, an antisense construct, a siRNA, an enzymatic RNA, a recombinant DNA construct, an expression vector, and mixtures thereof. The lipid vesicle delivery system of the present invention may be useful for in vivo or in vitro delivery of active agents, such as, amino acids, peptides and proteins. Peptides can be signalling molecules such as hormones, neurotransmitters or neuromodulators, and can be the active fragments of larger molecules, such as receptors, enzymes or nucleic acid binding proteins. The proteins can be enzymes, structural proteins, signalling proteins or nucleic acid binding proteins, such as transcription factors.

When the pharmaceutically active agent comprises a small organic active agent it may comprise a therapeutic agent or a diagnostic agent. In particular embodiments a small organic active agent may comprise a sequence-specific DNA binding oligomer, an oligomer of heterocyclic polyamides, for example, those disclosed in U.S. Pat. No. 6,506,906 which is hereby incorporated by reference. Other small organic active agents may comprise those disclosed by Dervan in “Molecular Recognition of DNA by Small Molecules, Bioorganic & Medicinal Chemistry (2001) 9:2215-2235”, which is hereby incorporated by reference. In certain embodiments, the oligomer may comprise monomeric subunits selected from the group consisting of N-methylimidazole carboxamide, N-methylpyrrole carboxamide, beta-alanine and dimethyl aminopropylamide.

In another embodiment of the invention, more than one type of polynucleotide may be enclosed within the lipid vesicle delivery system. Such polynucleotides provide the ability to express multiple gene products under control, in certain embodiments, at least one expressible gene product is a membrane protein, such as a membrane receptor, most preferably a membrane-bound receptor for a signalling molecule. In some embodiments, at least one expressible gene product is a soluble protein, such as a secreted protein, e.g. a signalling protein or peptide.

The present invention will now be described by way of example only, with reference to the accompanying Examples and Figures in which:

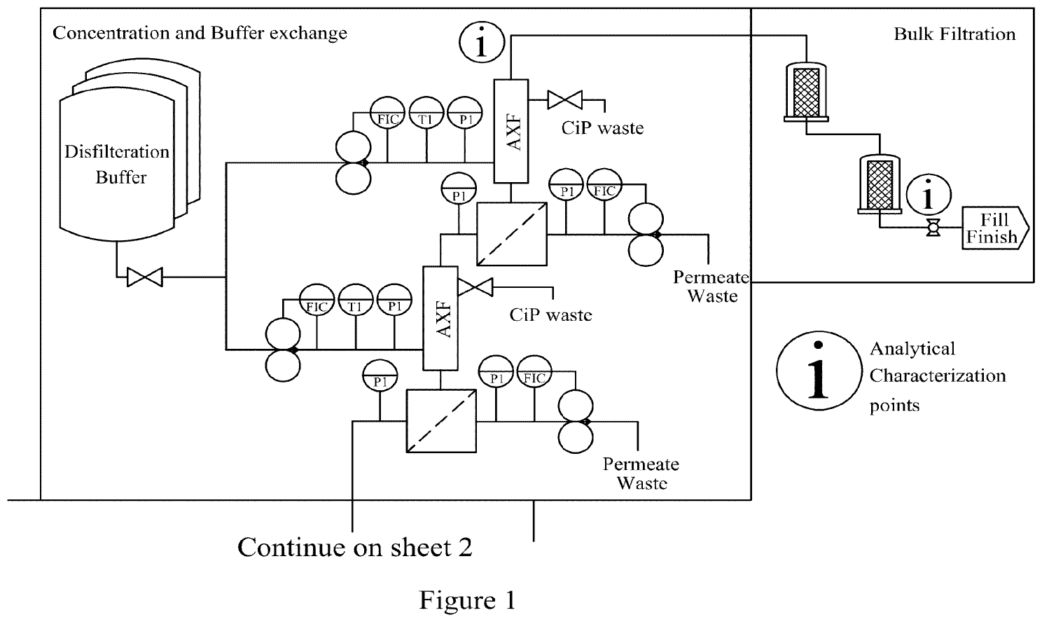

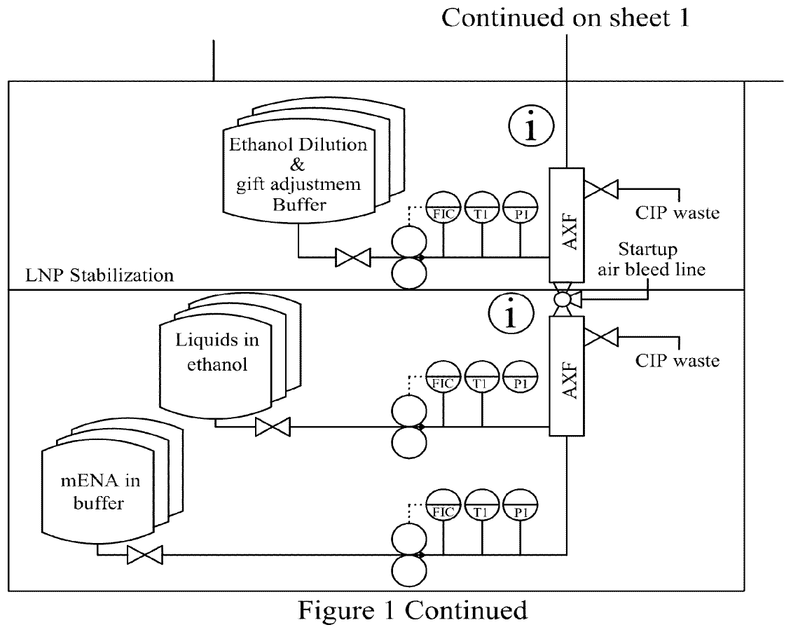

FIG. 1 illustrates a schematic representation of a continuous process of manufacturing LNPs, using Single-Pass Tangential Flow Filtration (SPTFF) membranes; and

FIG. 2 illustrates a schematic representation of a continuous process of manufacturing using slotted AXF® membranes for high flux microfiltration.

1. A continuous process for the preparation of microscopic particles, e.g. nanoparticles or microparticles, said process comprising the steps of:
(i) controlling provision of a first liquid phase to a first membrane, the first membrane defining a first plurality of apertures;
(ii) controlling provision of a second liquid phase to the first membrane via the first plurality of apertures to form a mixture;
optionally
(iii) controlling provision of the mixture to a second membrane, the second membrane defining a second plurality of apertures;
(iv) controlling provision of a pH buffer liquid phase to the second membrane via the second plurality of apertures to form a stabilised suspension of particles;
(v) concentrating the suspension of particles and controlling provision of the pH buffered mixture to a third membrane, the third membrane defining a third plurality of apertures;
(vi) controlling provision of a diafiltration buffering liquid phase to the third membrane via the third plurality of apertures to remove solvent and exchange the particles into an excipient buffer; and
(vii) controlling provision of the stabilised mixture to a fourth membrane for microfiltration, to isolate the microscopic particles;
wherein one or more analytical characterisation points are included.

2. A continuous process according to claim 1 wherein the microscopic particles are nanoparticles.

3. A continuous process according to claim 1 wherein the microscopic particles are microparticles.

4. A continuous process according to claim 1 wherein steps (i) to (vii) are carried out sequentially with or without an intervening isolation or purification step.

5. (canceled)

6. A continuous process according to claim 1 wherein each of the membranes, which may be the same or different, comprises a laboratory dispersion cell (LDC) or a crossflow apparatus (AXF®).

7. A continuous process according to claim 1 wherein the number of membranes used in the continuous process is from about 1 to about 4.

8. A continuous process according to claim 6 wherein at least one of the membranes comprises a crossflow apparatus (AXF®).

9. (canceled)

10. (canceled)

11. A continuous process according to claim 1 wherein in step (v) the concentration of the suspension of particles is achieved using single pass tangential flow filtration cassette equipped with an ultrafiltration or microfiltration membrane suitably sized to retain the particles.

12. (canceled)

13. A continuous process according to claim 1 wherein the process comprises the preparation of microscopic lipid vesicles.

14. A continuous process according to claim 13 wherein the lipid vesicles comprise liposomes.

15. A continuous process according to claim 13 wherein the lipid vesicles comprise lipid nanoparticles (LNPs).

16. A continuous process according to claim 13 wherein the process comprises the preparation of microscopic lipid vesicles comprising a lipid bilayer encapsulating an aqueous core.

17. A continuous process according to claim 15 wherein the process comprises the preparation of LNPs comprising an aqueous solvent phase core.

18. A continuous process according to claim 15 wherein the process comprises the preparation of LNPs comprising a non-aqueous solvent phase core.

19. A continuous process according to claim 16 wherein the aqueous core includes one or more active agents.

20. A continuous process according to claim 13 wherein the lipid vesicles are produced unloaded and loaded afterwards (active loading).

21. A continuous process according to claim 13 wherein the lipid vesicles are produced loaded (passive loading).

22. A continuous process according to claim 1 wherein step (iv) comprises formation of a pH buffered mixture by controlling provision of the mixture to a second membrane and controlling provision of a pH adjustment buffer to form a stabilised mixture.

23. A continuous process according to claim 1 wherein the microscopic particles are microparticles or nanoparticles including a chemically or biologically active substance.

24. A continuous process according to claim 1 wherein the microscopic particles are solidified particles, e.g. crystalline particles.

25. (canceled)

26. A continuous process according to claim 1 wherein an analytical characterisation point is included at or towards the end of each step of the continuous process or each pair of steps of the continuous process.

27. A continuous process according to claim 1 wherein an analytical characterisation point is included at or towards the end of one or more of steps (ii), (iv) and (vi).

28. A continuous process according to claim 8 comprising at least one crossflow membrane apparatus (AXF®) wherein said crossflow emulsification apparatus comprises:
an outer tubular sleeve provided with a first inlet at a first end; a lipid vesicle outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with a chamfered region; the chamfered region is provided with a plurality of orifices and a furcation plate; and
controlling provision of the first liquid phase to the tubular membrane; and controlling provision of a second liquid phase to the tubular membrane via the plurality of pores to form a lipid vesicle suspension.

29. A continuous process according to claim 28 wherein the crossflow apparatus includes an insert and the first inlet is a continuous phase first inlet and the second inlet is a disperse phase inlet; such that the disperse phase travels from outside the tubular membrane to inside.

30.-50. (canceled)
